(19) United States
(12) Patent Application Publication
(10) Pub. No.: US 2005/0245966 A1
(43) Pub. Date: Nov. 3, 2005

(54) Controlled viscosity tissue adhesive

(76) Inventors: Julius G. Hammerslag, La Quinta, CA (US); James V. Quinn, Portola, CA (US)

Correspondence Address: LEACH PATENT SERVICES, 5 EAST ALDINE DRIVE, HOCKESSIN, DE, 19707, US

(21) Appl. No.: 10/968,464

(22) Filed: Oct. 19, 2004

Related U.S. Application Data
(63) Continuation-in-part of application No. 10/145,581, filed on May 13, 2002, now abandoned.
Application No. 10/145,581 is a continuation of application No. 09/702,013, filed on Oct. 30, 2000, now Pat. No. 6,386,203.
Application No. 09/702,013 is a continuation of application No. 09/339,146, filed on Jun. 24, 1999, now Pat. No. 6,155,265.
Application No. 09/339,146 is a continuation-in-part of application No. 09/078,944, filed on May 14, 1998, now abandoned.
Application No. 09/078,944 is a continuation-in-part of application No. 08/991,823, filed on Dec. 17, 1997, now abandoned.

(51) Int. Cl. A61L 15/16

(52) U.S. Cl. 606/214

(57) ABSTRACT

Disclosed are methods and compositions for closing and sealing a wound, laceration, incision, or other percutaneous opening using an adhesive. In one preferred embodiment, the sides of the percutaneous opening are brought together in apposition and the adhesive is applied topically over the apposed opening and the skin adjacent thereto. Adhesives used in the methods of the preferred embodiments exhibit sufficient viscosity to substantially prevent flow of the adhesive into the percutaneous opening. Adhesives may also be used in surgical applications, as a covering for a trauma to the outer surface of the skin, or as a secondary means of closure in combination with other means of closure, including staples and sutures. In a preferred embodiment, the adhesive is a adhesive comprising cyanoacrylate monomer, cyanoacrylate polymer, and a plasticizer.

### CROSS-REFERENCE TO RELATED APPLICATIONS

This is a continuation-in-part of application Ser. No. 10/145,581, filed May 13, 2002, which is a continuation of application Ser. No. 09/702,013, filed Oct. 13, 2000, which is a continuation of application Ser. No. 09/339,146, filed Jun. 24, 1999, now U.S. Pat. No. 6,155,265, issued Dec. 5, 2000, which is a continuation-in-part of application Ser. No. 09/078,944, filed May 14, 1998, now abandoned, which is a continuation-in-part of application Ser. No. 08/991,823 filed Dec. 17, 1997, now abandoned, and also claims priority to U.S. Provisional Application Ser. No. 60/602,975, filed: Aug. 19, 2004, the disclosures of which are incorporated by reference herein in their entireties.

### BACKGROUND OF THE INVENTION

1. Field of the Invention

The present invention relates to medical and surgical tissue adhesives. In one embodiment, the adhesive is of the type useful for bonding adjacent sections of skin separated by percutaneous incision or traumatic injury.

2. Description of the Related Art

Every year, over 10 million traumatic wounds are treated by emergency physicians in the United States. A great many incisions ranging from a few millimeters to several centimeters in length are closed each year by medical personnel. Countless more less serious wounds are treated by non-medical persons, such as athletic trainers, parents of an injured child, or the injured individual himself.

Small wounds and lacerations can be treated by simply bandaging the wound or by using tape to keep the edges of the wound in apposition. Such methods can be performed with a minimum of time and training, as well as causing little or no additional trauma to the wound or causing the patient additional pain.

More serious wounds or incisions are generally treated by conventional methods, such as suturing. Suturing requires the use of a needle and often involves a local anesthetic. Suturing can be costly because it is time-intensive and the procedure requires that the individual performing it have some medical training. Additionally, suturing can be painful and the use of needles may cause further distress for an already traumatized patient, as well as expose medical personnel to potential needlestick injury. Furthermore, because most sutures used topically do not dissolve, the patient generally must make a return visit at a later date for the often uncomfortable procedure of removal of the sutures.

In recent years, cyanoacrylate tissue adhesives have been tried as an alternative for such conventional methods. The most commonly used cyanoacrylates for wound closure include octyl- and butylcyanoacrylate, have some advantages over suturing, such as faster and less painful closure. These monomers like all cyanoacrylate monomers have several drawbacks. One drawback is that they have a very low viscosity. The low viscosity makes precise application difficult, in that the adhesive flows over areas of the skin surface well beyond the immediate region of the closure and that the adhesive is readily drawn into the wound, effectively creating a barrier between the two tissue surfaces which are desirably rejoined in the natural healing process thereby blocking epithelealization and fibroblast growth. Furthermore, certain cyanoacrylate monomers and formulations form a closure which is hard, brittle, and inflexible, and which sets up too quickly to allow for adjustment of the opposing skin surfaces following its application.

### SUMMARY OF THE INVENTION

There remains a need for a simple and effective method and composition for effecting wound closure. Preferably, the method and composition can be utilized with minimal training time and risk of error, and will not materially increase complications, immunogenicity, scarring, infection, or other negative factors.

In accordance with one aspect, there is provided a tissue adhesive. The tissue adhesive comprises about 1-15% by volume cyanoacrylate polymer; about 2-15% by volume plasticizer and about 70-95% by volume of a mixture of first and second cyanoacrylate monomers, each having the general formula (I): 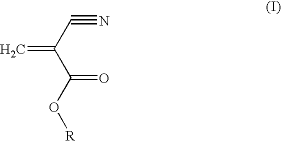
wherein R is alkyl and the first cyanoacrylate monomer has at least about 3 carbon atoms and the second cyanoacrylate monomer has no more than about 10 carbon atoms.

In accordance with another of the preferred embodiments, there is provided a method of closing a percutaneous opening, having a first dermal surface on a first side of the opening and a second dermal surface on a second side of the opening and generally coplanar with the first dermal surface. The method comprises applying an adhesive layer across at least a portion of the first and second dermal surfaces and spanning the opening, wherein the adhesive comprises a blended cyanoacrylate monomer, cyanoacrylate polymer and plasticizer; and exhibits a sufficient viscosity to substantially prevent flow of the adhesive into the opening.

In accordance with another preferred embodiment, there is provided a method of closing and sealing a joint formed between a tissue of a patient and a second surface, comprising positioning a tissue of a patient adjacent to a second surface; securing the tissue to the second surface using a primary closure modality to form a joint; delivering an adhesive comprising cyanoacrylate monomer, cyanoacrylate polymer, and a plasticizer to the joint; and permitting the adhesive to polymerize.

In accordance with a further embodiment, there is provided a method of closing and sealing a wound in a patient. The method comprises the steps of identifying a percutaneous wound having first and second sides, delivering a layer of wound closure adhesive comprising cyanoacrylate monomer, cyanoacrylate polymer, and a plasticizer to the surface of skin, blood vessel or other tissue on each of the first and second sides and across the wound in a quantity sufficient to retain closure and sealing of said would, and restraining the adhesive from entering the wound.

In accordance with a further embodiment, there is provided a method of covering a trauma to an outer layer of a skin in a patient. The method comprises delivering an adhesive comprising cyanoacrylate monomer, cyanoacrylate polymer, and a plasticizer to a surface of the skin spanning an area of the trauma to the outer layer of the skin in a quantity sufficient to cover said trauma; and permitting the adhesive to polymerize.

Further features and advantages of the present invention will become apparent to those of skill in the art in view of the detailed description of preferred embodiments which follows, when considered together with the attached drawings and claims.

### BRIEF DESCRIPTION OF THE DRAWINGS

FIG. 1 is a cross-section of a rollerball container-applicator as can be used to apply adhesives in accordance with the preferred embodiments.

FIG. 2 depicts the use of a rollerball container-applicator of the type in FIG. 1 to deliver adhesive to a topical wound to effect closure in accordance with the preferred embodiments.

FIG. 3 is a view of an alternate container-applicator for use in accordance with the preferred embodiments.

FIG. 4 is a cross-section of the container-applicator of FIG. 3.

FIG. 5 is a blown-up view of the applicator tip of the container-applicator of FIG. 3 showing the placement of a break-away sealing tip.

FIG. 6 is a cross-section of a percutaneous opening or wound which has been closed and sealed according to the preferred embodiments.

FIG. 7 is a view of a tube container-applicator for use in accordance with the preferred embodiments.

### DETAILED DESCRIPTION OF THE PREFERRED EMBODIMENTS

Several considerations come into play when a closing percutaneous opening, such as a wound or incision. The considerations include providing a closure having adequate strength to resist opening or rupture and providing a closure which protects the opening, but does not at the same time substantially interfere with the normal healing processes. One method that can be used is the application of an adhesive. An adhesive can be used either with or without additional closure means.

When the adhesive used is a liquid, it presents a different set of considerations as compared to solid materials and other conventional methods of closure, such as staples, sutures, and bandages. Several of these considerations have to do with the viscosity of the adhesive.

In discussing the viscosity of the adhesives in the context of preferred embodiments, the viscosity referred to herein is the viscosity of the adhesive at the time it is being applied. Following application, the adhesive will increase in viscosity until the adhesive “sets up” to form the final solid or relatively solid state of the adhesive closure due to physical or chemical mechanisms in the adhesive or adhesive preparation, including, but not limited to, curing, cross-linking, polymerizing, and evaporation of solvent. Once the adhesive has set up to form the closure, it will preferably take a solid form, which can be flexible, rubbery or stiff, with firm but flexible closures being preferred. Furthermore, unless specifically referenced otherwise, all percentages herein are percentages by volume.

Adhesives used in accordance with preferred embodiments preferably have a viscosity low enough such that they flow (and can be spread) when acted upon by gravity or some other force, such as being squeezed out of a tube or spread with an applicator. This action allows for the adhesive to wet the skin adjacent the opening and also allows for application of the adhesive by a variety of methods. On the other hand, the viscosity of the adhesive during application is preferably not so low that the adhesive becomes runny and flows far beyond the general vicinity of the intended application surface or that it flows into the opening itself. This is an especially important consideration, because if the adhesive flows a substantial distance into the opening, it can block the surfaces that are to be healed together, and thus can actually impede or prevent the healing process by acting as a barrier to the migration of basal cells and collagen in the natural healing of a wound. Seepage into the wound is a significant problem with adhesives known in the art, such as liquid cyanoacrylates which have a very low viscosity and will, when placed on a wound, run into the wound or be drawn therein via capillary action. Accordingly, a preferred adhesive polymerizes and sets before it runs into a wound, incision, opening, or into undesired areas. An example of a preferred situation of applying an adhesive is applying a layer of the adhesive to a horizontal wound plane and then moving the wound plane to a vertical position and allowing the adhesive to polymerize without running.

To achieve at least some of the properties discussed above, the adhesive used to close a percutaneous opening in accordance with the preferred embodiments preferably has sufficient viscosity, realizing that “sufficient viscosity” is a combination of the volume and mass of material used as well as the setting times of the adhesive used to achieve the purpose. In general this is a viscosity greater than about 100 centipoise, but may be as low as 50 cps if initiators or accelerators are use to speed the rate of curing so that the adhesive sets before flowing into unwanted areas. Although very high viscosity materials can be used in accordance with the preferred embodiments, viscosities of less than about 100,000 are generally used, and it is preferred that the viscosity be less than about 5,000 centipoise, or less than about 2500 cp such that the adhesive maintains a reasonable amount of workability and ability to flow under pressure. In a preferred embodiment, the viscosity of the adhesive is within the range of from about 100 to about 10,000 centipoise, including about 200 cp, 300 cp, 400 cp, 500 cp, 600 cp, 700 cp, 800 cp, 900 cp, 1000 cp, 2000 cp, 3000 cp, 4000 cp, 5000 cp, and 7500 cp. Ranges of viscosities which comprise the preceding viscosities and those between the recited viscosities and are limited at their high and low ends by any two of the recited viscosities are also contemplated (e.g. 300-700 cp, 400-900 cp, etc.).

Adhesives according to preferred embodiments preferably comprise cyanoacrylates. Cyanoacrylates have the general formula as shown below. As also shown below, cyanoacrylates can polymerize to polycyanoacrylates in the presence of water from the air or trace amounts of moisture on the surface to which the adhesive is being applied. Upon application to the surface, the monomer undergoes an exothermic hydroxylation reaction that results in polymerization.

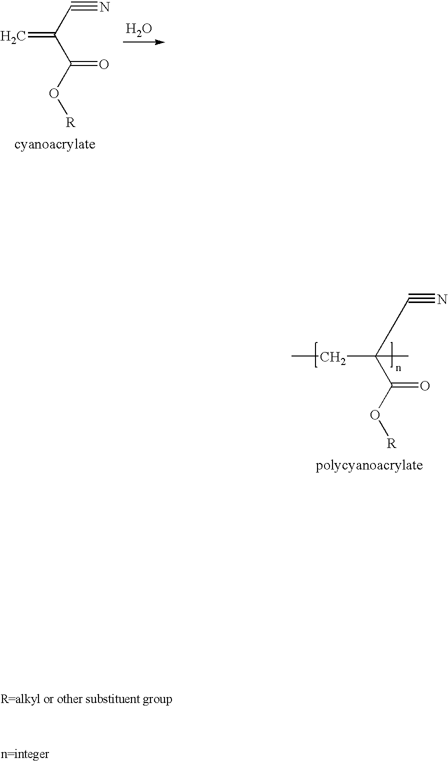

In the formula above, R may be any organic group which does not interfere with the polymerization of the monomer to form the polycyanoacrylate. In a preferred embodiment, R is alkyl which includes straight-chain, branched, and cyclic groups, and include from about 2 to about 12 carbon atoms, more preferably from about 3 to about 10 carbon atoms.

A cyanoacrylate can been modified to increase its viscosity and/or decrease its polymerization rate. The viscosity of the cyanoacrylate can be increased to a gel or paste form by chemical modification of the cyanoacrylate molecule and/or by the presence of one or more viscosifying agents.

Examples of compounds used in connection with certain embodiments include polymerizable cyanoacrylates that have been cross-linked or co-polymerized with other compounds that can alter elasticity, modify viscosity, aid biodegradation or change some other property of the resulting material. For example, polyacrylic acid having a molecular weight of 200,000 to 600,000 can be cross-linked to a cyanoacrylate to form compounds that allow the absorbability to be coordinated with the tissue regeneration rate and can feature higher elasticity than cyanoacrylates alone. Absorbability is generally unnecessary for topical applications, with the adhesive film simply falling off in a few days.

In a preferred embodiment, the adhesive comprises the following components: 1) cyanoacrylate monomer, 2) cyanoacrylate polymer, and 3) plasticizer. The cyanoacrylate monomer gives adhesive properties. In the choice of a cyanoacrylate monomer for adhesiveness, factors to be considered include rate of polymerization, tensile strength, and brittleness and flexibility. These factors are also balanced with the choice of the other ingredients, the cyanoacrylate polymer and plasticizer. The cyanoacrylate polymer which, having a high molecular weight, is used as a viscosifying agent and adds flexibility. The cyanoacrylate polymer also gives tensile strength. The plasticizer enhances flexibility and resilience. Additionally, to the formulations of the preferred embodiments, any of a variety of other additives can also be added, such as bacteriostatic agents, anti-inflammatory agents, and preservatives, stabilizers and the like, as will be understood by those of skill in the art. In some embodiments, formulations of tissue adhesives can additionally comprise one or more optional additives, such as polymers, viscosity modifiers, colorants, perfumes, anti-diffusion agents, salts, antibiotics, anti-microbials, stabilizers, desiccants, catalysts, or agents that alter polymerization rate.

Among the reasons that cyanoacrylates are preferred for use in tissue adhesives are their several particular advantages as an adhesive compound. First, they can harden almost instantaneously on contact with surfaces having moisture thereon. These contact areas include most tissues and surfaces in and on the body of an animal, such as a human. Second, experiments by the inventor indicate that cyanoacrylate sealed vascular punctures can withstand several times the maximum potential systolic pressure, and hence, would not be expected to fail when used to seal most surface wounds. Cyanoacrylates are also naturally thrombogenic. This property is an advantage in certain applications as it promotes the first step in healing.

Preferred compounds to be used in the adhesives disclosed herein include at least one of the biologically suitable monomer compounds within the cyanoacrylate family. The cyanoacrylate family includes methyl cyanoacrylate, ethyl cyanoacrylate, n-propyl cyanoacrylate, isopropyl cyanoacrylate, n-butyl cyanoacrylate, isobutyl cyanoacrylate, n-amyl cyanoacrylate, isoamyl cyanoacrylate, hexylcyanoacrylate, octylcyanoacrylate, 3-acetoxypropyl cyanoacrylate, 2-methoxypropyl cyanoacrylate, 3-chloropropyl cyanoacrylate, benzyl cyanoacrylate, phenyl cyanoacrylate, alkenyl cyanoacrylate, butyl-2-cyanoacrylate, alkoxyalkyl 2-cyanoacrylates, fluorinated 2-cyanoacrylates, and carbalkoxyalkyl cyanoacrylates, depending upon acceptable toxicity and other properties for a given application. In a preferred embodiment, the adhesive compound comprises octylcyanoacrylate and butylcyanoacrylate. Other members of the cyanoacrylate family may be commercially available or can be synthesized according to published procedures or analogous methods as is within the abilities of one skilled in the art.

Among the preferred cyanoacrylate monomers are octylcyanoacrylate, in which R is octyl, and butylcyanoacrylate, in which R is butyl. The properties of the cyanoacrylate can be modified by altering the R group of the alkoxycarbonyl group. The shorter-chain derivatives tend to have a higher degree of tissue toxicity than the longer-chain derivatives. However, the shorter-chain derivatives provide advantages as a dermal adhesive according to its properties. Shorter-chain derivatives provide stronger bonds and more rapid curing. For example, polybutylcyanoacrylates are rigid when dry, but provide a strong bond and polyoctylcyanoacrylates are more flexible when dry, but produce a weaker bond. However, the shorter-chain derivatives provide advantages as a dermal adhesive according to its properties. Shorter-chain derivatives provide stronger bonds and more rapid curing. For example, polybutylcyanoacrylates are rigid when dry, but provide a strong bond and polyoctylcyanoacrylates are more flexible when dry, but produce a weaker bond.

Inflammation, tissue necrosis, granulation formation, and wound breakdown can occur when cyanoacrylates are implanted subcutaneously. The process causing the histological toxicity is thought to be related to a general foreign body reaction and the by-products of degradation, cyanoacetate and formaldehyde. The local concentrations of these breakdown products are proportional to the rate of degradation (an aqueous degradation process) of the parent compound. Therefore, slower degradation rate results in less toxicity to the tissues. This phenomenon is explained by the hypothetical possibility that slowly degrading compounds release degradation products more gradually, thereby permitting more effective clearance and invoke a less intense inflammatory response. The longer-chain compounds degrade much more slowly than the shorter-chain compounds, hence the lower reactivity and toxicity of the longer-chain compounds.

There is a wide variation in the rates and facility of in vivo biodegradation of polymers made from cyanoacrylate monomers which can be used as adhesive compounds in the preferred embodiments and this wide variation is contemplated. Generally, polymers of cyanoacrylates which have substituents that are small and/or contain one or more oxygen-containing functional groups (e.g. ether, ester, carbonyl) appear to have increased biodegradability rates. Cyanoacrylates having long chain alkyl groups lacking in oxygen-containing functional groups as substituents can tend to form polymers which biodegrade more slowly. There are also indications in the literature that the biodegradation rate of cyanoacrylate polymers is affected by the polymer molecular weight and crystallinity of the polymer.

There are several studies of biodegradation rates of polymers formed by various members of the cyanoacrylate family in the scientific and medical literature. It is within the abilities of one of skill in the art to use such information in the literature along with routine experimentation in order to choose a member of the cyanoacrylate family with suitable biodegradation characteristics for use in accordance with the preferred embodiments.

The above-listed members of the cyanoacrylate family, as well as other members of the cyanoacrylate family and other adhesive compounds that fall within the scope of the preferred embodiments and are not listed above, can differ in their properties when used in a adhesive. The efficacy, histotoxicity, and other medically relevant properties of above-listed and other members of the cyanoacrylate family can be readily determined by routine experimentation by one of ordinary skill in the art or by review of the medical literature. Such experimentation will enable one skilled in the art to choose optimal cyanoacrylate or other adhesive compounds for use in the adhesive of the preferred embodiments for a desired specific application.

In preferred embodiments, cyanoacrylate polymer is used as a viscosifying agent. A long chain cyanoacrylate polymer can also aid in overcoming brittleness of the adhesive material. Also among the properties that change with the quantity of cyanoacrylate polymer used are viscosity and polymerization rate. Increasing the percentage of cyanoacrylate polymer in the adhesive will increase the viscosity of the adhesive. Increased viscosity provides for easier application of the adhesive on tissues, as viscous adhesives stay where they are placed and thus decrease the incidence of running or dripping onto other surfaces or tissues, or leaking in between sides of an opening to be closed. Decreased polymerization rates allow more time for a practitioner to place and adjust the surfaces that are to be sealed. Where the polymerization time is short, there can be little if any margin for error before the surfaces are sealed. The longer polymerization time has an additional benefit. Since the polymerization process is exothermic, decreasing the polymerization rate decreases the rate that heat is released by the adhesive, resulting in a lower temperature in the adhesive and surrounding tissues during polymerization.

Preferred cyanoacrylate polymer compounds to be used in the viscosifying agent of the preferred embodiments include a biologically suitable polymer compounds made from at least one member within the cyanoacrylate family. As stated above, the cyanoacrylate family includes methyl cyanoacrylate, ethyl cyanoacrylate, n-propyl cyanoacrylate, isopropyl cyanoacrylate, n-butyl cyanoacrylate, isobutyl cyanoacrylate, n-amyl cyanoacrylate, isoamyl cyanoacrylate, hexylcyanoacrylate octylcyanoacrylate, decylcyanoacrylate, 3-acetoxypropyl cyanoacrylate, 2-methoxypropyl cyanoacrylate, 3-chloropropyl cyanoacrylate, benzyl cyanoacrylate, phenyl cyanoacrylate, alkenyl cyanoacrylate, butyl-2-cyanoacrylate, alkoxyalkyl 2-cyanoacrylates, fluorinated 2-cyanoacrylates, and carbalkoxyalkyl cyanoacrylates, depending upon acceptable toxicity and other properties for a given application. Other members of the cyanoacrylate family may be suitable to form the cyanoacrylate polymer for use as a viscosifying agent. Generally speaking, cyanoacrylate polymers having longer chain R groups are preferred. Examples of cyanoacrylate polymers having a longer chain R group are poly(octylcyanoacrylate) and poly(decylcyanoacrylate).

Another component in preferred embodiments of tissue adhesives is a plasticizer. Preferably, the plasticizer is suitable for use in the mammalian body. Examples of suitable plasticizers include, but are not limited to, tributyl citrate, acetyl tributyl citrate, dimethyl sebacate, diethylsebacate, triethyl phosphate, tri(2-ethyl-hexyl)phosphate, tri(p-cresyl) phosphate, glyceryl triacetate, glyceryl tributyrate, dioctyl adipate, isopropyl myrisate, butyl sterate, lauric acid, trioctyl trimelliate, dioctyl glutatrate and mixtures thereof. Tributyl citrate is preferred.

In a preferred embodiment, the adhesive comprises about 70-95% cyanoacrylate monomer by volume, including 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, and 94%; about 2-15% cyanoacrylate polymer, including about 3%, 4%, 5%, 6%, 7%, 8%, 9%, 10%, 11%, 12%, 13%, and 14%; and about 3-15% plasticizer, including about 4%, 5%, 6%, 7%, 8%, 9%, 10%, 11%, 12%, 13%, and 14%. Ranges of percentages which comprise the preceding percentages and those therebetween and are limited at their high and low ends by any two of the recited percentages are also contemplated. For example, an adhesive may comprise about 80-90% cyanoacrylate monomer, about 3-10% cyanoacrylate polymer, and about 5-10% plasticizer. In another embodiment, an adhesive comprises about 84% cyanoacrylate monomer, about 8% cyanoacrylate polymer, and about 8% plasticizer.

In a preferred embodiment, the cyanoacrylate monomer component comprises one or more cyanoacrylate monomer. By blending monomers, the properties of the adhesive can be modified. Cyanoacrylates with longer R groups can take a longer time to polymerize; can have more flexibility; can have slower degradation; and can have lower tensile strength. Also, cyanoacrylates with longer R groups often utilize initiators to set or cure. On the other hand, cyanoacrylates with shorter R groups can set very quickly and release a significant amount of heat upon setting. Hence, cyanoacrylates with shorter R groups may utilize stabilizers to offset the quick setting and significant heat release. Also, despite great tensile strength, cyanoacrylates with shorter R groups tend to polymerize to produce a brittle material and are prone to cracking and premature sloughing when used topically on the skin. By combining a cyanoacrylate with a longer R group with a cyanoacrylate with a shorter R group into blends, the properties of the resulting adhesive can be modified. Although blends comprising one or more cyanoacrylates having longer R groups and one or more cyanoacrylates having shorter R groups are preferred in some embodiments, blends may include only those having longer R groups or only those having shorter R groups.

One preferred cyanoacrylate monomer component composition comprises octylcyanoacrylate, as a cyanoacrylate with a longer R group, and butylcyanoacrylate, as a cyanoacrylate with a shorter R group. Such compositions preferably comprise about 50% to about 95% octylcyanoacrylate and about 5% to about 50% butylcyanoacrylate, including compositions having the following ratios of octylcyanoacrylate to butylcyanoacrylate: 50:50, 55:45, 60:40, 65:45, 70:30, 75:25, 80:20, 85:15, 90:10, and 95:5. Ranges of composition ratios which comprise the preceding ratios and those therebetween and are limited at their high and low ends by any two of the recited ratios are also contemplated. One of ordinary skill of the art can optimize the properties of the cyanoacrylate monomer component by routine experimentation with the amounts of the members of the cyanoacrylate monomer component. In a preferred embodiment, mixtures having higher levels of cyanoacrylates having longer chain R groups further comprise an initiator to increase the speed at which the adhesive sets up or cures. In the case of octylcyanoacrylate, use of an initiator is preferred in those embodiments having greater than about 80% octylcyanoacrylate.

In another embodiment, the cyanoacrylate monomer component comprises a single cyanoacrylate monomer, with those having longer chain R groups being preferred.

Depending upon the nature of the placement and composition of the two surfaces to be joined, the degree of biodegradability or bioabsorbability desired in the adhesive employed can vary. For wounds or incisions on the surface of the skin, it can be acceptable to use a adhesive that is only slowly degradable or substantially nonbiodegradable. For example, it is preferable that the adhesive material would slough off the surface of the skin in about 7 to about 10 days. Alternatively, if both surfaces are living tissues which are internal, it may be preferable to use an adhesive that will biodegrade over a period of days or weeks, diminishing as the natural healing mechanisms take hold.

If an adhesive having a chemical composition suitable for use in wound closure does not have a viscosity in the preferred range, the preferred working viscosity can be achieved in a variety of ways. If the desired adhesive has a higher viscosity, such as can be found with a thick gel or rubber-like material, the adhesive can be combined with a solvent of high or moderate volatility to lower the viscosity into the preferred range. The solvent could then evaporate when it comes into contact with the warm surface of the skin.

For thinner materials, which will likely form the great bulk of desirable adhesives, the viscosity should preferably be increased. If an adhesive sets up by means of polymerizing, cross-linking or other curing mechanism, a partially cured adhesive preparation can be used. By using a partially cured adhesive, the viscosity could be brought within a suitable range for application according to the discussion herein. Such an adhesive can be prepared by initiating the curing mechanism and then quenching it, such as by adding an inhibitor. The curing mechanism would then need to be re-initiated prior to application, or immediately thereafter. This type of method could be used for UV-curable adhesives, for which re-initiation could begin by means of a UV lamp or natural sunlight once the adhesive is removed or expressed from its container. This method would also be suitable for adhesives which set up in the presence of water, in that moisture in the air or on the skin could provide the needed water, or the site could be swabbed with water prior to application.

Although specific closure means and support structures are identified and discussed in this specification, such use of the terms should not be construed as limiting the definitions of these terms. It is the applicant's intention that these terms be given their broad ordinary meanings.

An adhesive according to preferred embodiments can be used to effect wound or percutaneous incision closure in a manner that is quick, simple, and effective. The preferred embodiments provide for a method of closing a percutaneous opening, having a first dermal surface on a first side of the opening and a second dermal surface on a second side of the opening and generally coplanar with the first dermal surface. The method comprises a step of applying an adhesive layer across at least a portion of the first and second dermal surfaces and spanning the opening. The materials and methods of the preferred embodiments require little training for their use and can be used by medical personnel to replace conventional methods of closing wounds. Additionally, they can be used by non-medical persons for use in combination with or as a replacement for conventional home remedies, such as adhesive bandages.

In accordance with preferred embodiments, an adhesive is used to join adjacent surfaces of skin to effect closure of a wound or incision. The adhesive used in accordance with the preferred embodiments are typically used as the primary closure modality, to replace sutures or staples. The two sides to a percutaneous incision, for example, can be held together and a layer of adhesive can be placed on the surface to span the incision. After sufficient polymerization, the adhesive will provide a strong bond while natural healing processes occur under the surface. It is preferred that the adhesives of the preferred embodiments are used as the primary method of wound closure, but they can be used in conjunction with other wound closure or tissue fastening systems, such as staples and sutures, or in combination with a support structure such as cloth or gauze.

Especially preferred adhesives are those which have a viscosity such that, when it is placed on the skin, the adhesive will span the gap between the two or more surfaces of the opening with no flow into the opening or without flowing a substantial distance into the opening. The opening spanned without substantial seepage into the wound is generally about 0.1 mm to about 4 mm wide, preferably 0.5 mm to about 1.5 mm wide, and for many applications about 1 mm wide.

When adhesive is used having sufficient viscosity to span an opening as described above, the adhesive can be made to form a thicker layer above the wound than would be possible with a thinner adhesive. This is because the more viscous adhesive will have a greater resistance to flow under its own weight and will thus be more likely to stay in a shape closely approximating that in which it was applied. In one preferred embodiment, the adhesive, when applied to the skin, has the profile seen in FIG. 6. Such a profile can be achieved by using an applicator such as that shown in FIGS. 3 and 4 or by applying a generally rounded bead of adhesive to the skin which then wets the skin surface. This profile of adhesive on the skin, wherein the layer of adhesive 18 forming the closure is relatively thicker in the area generally over the opening 20 between the pieces of skin 19, has advantages in that a thicker layer of a given adhesive will have greater tensile strength than will a thinner layer of the adhesive. The increase in tensile strength of the material forming the closure will provide increased protection against tearing or rupture of the opening following sealing.

In addition to the foregoing, the adhesive materials may be used as a secondary closure modality, being used in combination with sutures, staples, and other such primary closures. In such methods, the adhesive is applied to the surface of the wound or incision, preferably after the wound is closed using the chosen primary closure means. The use of an adhesive further strengthens the closure and can also seal and protect the closure area.

The adhesive material according to the preferred embodiments can also be used as a dressing to cover any trauma to the outer layer of the skin. Trauma to the outer layer of skin includes, but is not limited to, abrasions, scrapes, burns, blisters, bedsores, ulcers, chapping, and chafing. The adhesive material is delivered to a surface of the skin covering an area of the trauma to the outer layer of the skin in a quantity sufficient to substantially cover the trauma, or some portion thereof, and then is permitted to polymerize or cure allowing healing underneath the polymerized material.

In addition to using the adhesive material for closure and sealing of wounds and as coverings for trauma to the outer layer of the skin, the adhesive material may also be used as a surgical adhesive to bind tissues within the body to other tissues or materials. In such methods, a first surface and second surface are joined using the adhesive material. A first surface to be joined may be a surface of tissue in the patient, and the second surface to be joined may be an autologous tissue from elsewhere in the same patient such as a harvested vessel, allograph tissue such as a human transplant organ from a separate donor, animal tissue such as porcine or bovine heart valves, which may have been pretreated in accordance with known technology, or any of a wide variety of nontissue materials. Nontissue materials suitable for use with the preferred embodiments include any of a variety of biologically compatible metals such as stainless steel, gold, platinum, or others well known in the medical device industry. The second surface may also be any of a wide variety of polymeric materials, including polyethylenes, polypropylenes, nylons, polytetrafluoroethylene and other polyfluoro compounds, polyesters such as Dacron, and other polymeric materials known in the art. The second surface may also be a surface on any of a wide variety of implantable prostheses, prosthetic devices, grafts or organs. For example, the adhesive material may be useful in heart transplant, kidney transplant, liver transplant, lung transplant or other transplant procedures. In addition, a wide variety of vascular grafting procedures can benefit from the use of adhesives according to preferred embodiments, including tissue grafts using vessels harvested from elsewhere in the patient's body, as well as prosthetic grafts of the type which may be made from PTFE, Dacron, or other materials, either alone or in combination with supporting metallic cages. In addition, the adhesives may be used in connection with the implantation of replacement heart valves which will be subject to arterial pressure. Such valves may include mechanical valves, bioprosthetic valves, and human allografts. Although specific prostheses, prosthetic devices, grafts and organs are named and discussed herein, such use of the terms should not be construed as limiting the definitions of these terms. It is the applicant's intention that these terms be given their broad ordinary meanings. Additionally, the terms device, graft, prosthesis and organ should be interpreted as including any skirts, supports, coverings or additional materials attached to or associated with the device, graft, prosthesis or organ.

In addition to the foregoing, the adhesive material may also be used for skin, cartilage and bone grafting, sealing cerebrospinal fluid leaks, Tympanoplasty, ossiculoplasty, sealing bowel and vascular anastomosis, sealant for fractured teeth, and dressing for Aphthous ulcers.

The surgical method proceeds by bringing together the two surfaces that will form the joint and initially securing them together by a primary closure, including, but not limited to, those formed by the use of sutures, staples, or other materials and methods known in the art. The joint between the two surfaces is then sealed and further secured by applying the adhesive to the interface of the two surfaces. The method of application may, in part, be determined by factors such as the characteristics of the chosen adhesive and the geometry, size and placement of the application site. If required, the two surfaces are held together by use of a suitable surgical instrument for the time required for polymerization of the adhesive.

Alternatively, the two (or more) surfaces may be brought together by clamps, forceps, hands or other removable means and secured and sealed by means of the adhesive alone, with the adhesive being applied in a quantity sufficient to retain closure or attachment of the surfaces. The adhesive may be applied to one or both surfaces, either before or after the surfaces are brought together. After polymerization to the point of adhesion, the clamps, forceps or the like are then removed such that the adhesive is the primary or even the only securing modality.

In other methods, the adhesive material is applied directly to a small opening, such as a puncture, in a quantity sufficient to close and seal the opening. In the case of a very small opening, the adhesive material can close the opening without any need for a separate step of bringing together two or more surfaces to be secured together.

Preferably, the adhesive material is allowed to dry, polymerize and/or cure following its application. The polymerization rate of the adhesive material should be such that the time to set the adhesive material should not be too short or too long and suitable for the clinical indication. A short polymerization time would allow little if any margin for error for sealing surfaces. A long polymerization time would not allow the convenience of a quick-sealing adhesive. Hence, a preferred set-up time for the adhesive material is about 10 to about 180 seconds, more preferably about 15 to about 90 seconds, including 20, 30, 40, 50 and 60 seconds. The curing time is the time for the adhesive material to obtain maximal strength and completely dry and polymerize. In one embodiment, preferred curing times for the adhesive material are about 1 to about 5 minutes, including about 2, 3 and 4 minutes.

What follows is a discussion of several applicators and methods of applying the adhesives of the preferred embodiments. It is contemplated that any of the adhesives described herein can be used in connection with the devices and methods described as well as with other devices and methods as will be apparent to those skilled in the art.

Any of a variety of containers, made of materials such as glass, plastic or aluminum, or devices can be used to apply or deliver adhesive to a wound for closure and sealing. For example, syringes, eyedroppers, compressible bottles or tubes, tongue depressors, spatulas, and the like can be used to deliver adhesive to the site intended for sealing. Adhesive can also be applied manually without the use of an applicator. Additionally, devices designed to deliver sealing adhesive can be used, such as that disclosed in U.S. Pat. No. 5,529,577. Once they are formulated, adhesives may be placed in container-applicators such as those discussed in greater detail below. The choice of application or delivery means can, in part, be determined by the viscosity of the adhesive employed. The choice of delivery means can also depend on other factors, such as the nature, physical structure, and location on the body of the wound, incision or other opening to be closed and sealed.

Proper storage of tissue adhesives is an important consideration. For example, if a UV-curing adhesive is used, the storage container preferably prevents penetration of UV radiation, and if a water-curing adhesive is used, a desiccant may be used. Because many cyanoacrylates will polymerize and harden relatively rapidly when stored below a critical volume, it is preferable for the vessel or reservoir in which the adhesive is stored to contain more adhesive than is necessary to seal a typical site. Preferably, the storage vessel or reservoir in a single-use container or container-applicator will contain a minimum of about 0.2 to about 5 grams of adhesive or more to maintain the cyanoacrylate monomer component in a generally unpolymerized state in the storage vessel or reservoir prior to use. For multiple-use containers or container-applicators, the reservoir preferably contains about 1 to about 10 grams, more preferably about 3 to about 5 grams of adhesive. In certain embodiments, the volume of the container is half filled to help maximize shelf life. The total volume of adhesive, the desiccation measures, and the sealing structures in the container or container-applicator can be optimized by one of skill in the art to provide enhanced shelf life.

The tissue adhesives of the preferred embodiments are preferably stored and applied using a container-applicator. A container-applicator has two basic parts: (1) a storage area or reservoir which holds the adhesive and protects it from air, water and contaminants; and (2) the applicator which comprises a specially shaped tip designed to aid in application of adhesive.

The reservoir is preferably both air-tight and water-tight, and keeps the adhesive within free from contaminants. The reservoir can contain a desiccant material to keep the adhesive free of water, which would cause polymerization of the preferred cyanoacrylate-based adhesive. Reservoirs can be of any shape, although shapes which provide for a smooth internal flow of adhesive, such as cylindrical or pyramidal shapes, are preferred. The size of the reservoir can vary within a wide range, but is preferably slightly larger than the volume of adhesive which will be placed inside the reservoir to minimize the amount of gas within the reservoir. The reservoir can be made from any of a variety of medical grade materials, such as plastics, that is suitable for the storage of cyanoacrylates as is known in the art. The reservoir can be either rigid, collapsible, or compressible. Use of a compressible or collapsible reservoir allows the user to have greater control over the rate at which adhesive is expressed, as exertion of pressure on a compressible or collapsible reservoir would place a force on the on the adhesive causing it to flow at a faster rate than it would in the absence of such pressure. The compressible or collapsible reservoir design is especially preferred for highly viscous or gel-like adhesive for which the force of gravity may not be strong enough to cause a flow of adhesive through an applicator sufficient to close a wound. Collapsible reservoirs which retain their collapsed shape have the additional advantage of reducing the amount of air which enters the reservoir following use. This advantage of collapsible containers is of greater importance in multiple-use (reusable) devices, wherein adhesive is preferably kept relatively free of potential contaminants between uses.

Applicator tips can be of any of a number of shapes, sizes, and configurations. They are preferably fairly rigid and may be made out of any material which is compatible with the adhesive formulation, and may be plastic, cotton tipped swabs or high density foams, preferably plastic. The choice of a proper applicator tip for a given application will depend on factors such as the viscosity of the adhesive, the desired application rate of the adhesive, the nature of the wound, the placement of the wound on the body, and the physical structure of the wound.

The container-applicators of the preferred embodiments can be either single-use or multiple-use devices. For most applications, single-use container-applicator devices are preferred. This preference arises because the risk of cross-contamination between wounds or patients is practically eliminated when a new device is used for each closure. As an alternative to the single-use embodiment, a container or reservoir containing enough adhesive for multiple closures may be configured to accommodate replaceable tips. In such an embodiment, at the place whereon the replaceable tips connect with the reservoir, the reservoir would preferably have a means such as a valve, septum or sealing gasket which allows the reservoir to be sealed in the absence of an applicator tip. Placing an applicator tip on the reservoir would cause the valve to open, allowing adhesive to flow out from the reservoir. In this manner, one reservoir containing enough adhesive to close several wounds could be used over a period of hours, days or weeks. This embodiment would also allow the user to use one reservoir with applicator tips of varying shapes and sizes chosen to best accommodate the needs of different wounds.

Three specific embodiments of container-applicators are depicted in the drawings and detailed below.

One preferred embodiment of container-applicator is the rollerball container-applicator 1 depicted in FIG. 1. The reservoir 2 can be either rigid, compressible, or collapsible and can be made out of any material suitable for the storage of cyanoacrylates, as is known in the art. The applicator tip portion of the container-applicator comprises a ball 3 and a cuff 4. The ball 3 is held loosely within the cuff 4 so that the ball 3 is free to rotate in any direction, but not so loosely as to allow the ball 3 to be removed or fall out when the container-applicator 1 is inverted. The size of the gap 5 formed between the ball 3 and the cuff 4 can be varied to accommodate a wide range of viscosities of adhesive and desired flow rates. For low viscosity adhesives, a relatively small gap 5 would be preferred to allow the adhesive to flow out around the ball at a reasonable rate during application, whereas for high viscosity gel-like adhesive a larger gap 5 would be required to allow a reasonable flow of adhesive around the ball 3. Similarly, the gap 5 can be varied to achieve a desired application rate for adhesives of a particular viscosity. For adhesive of a given viscosity, a large gap 5 would provide a higher flow rate for the adhesive than a smaller gap 5. Furthermore, use of a compressible or collapsible reservoir 2 allows for additional control over the rate at which adhesive is expressed, as exertion of pressure on the compressible reservoir increases the pressure on the adhesive causing it to flow through gap 5 at a rate faster than that for the same adhesive in the absence of exerted pressure, regardless of viscosity.

A second embodiment of container-applicator is that depicted in FIG. 3. This embodiment comprises a reservoir 11 and an applicator tip 12. The container-applicator 10 can further comprise a one-time removable or breakable sealing tip or cap as described below. In the illustrated embodiment, the adhesive flows from the reservoir 11 through a tubular extension 12 and out to the application site through an opening 13 in the distal end of the applicator tip 14. In one preferred embodiment, the length of the extended portion 12 of the applicator tip 14 is preferably about 0.1 to about 10 cm long, more preferably about 0.5 to about 2 cm, but can be readily optimized in view of an intended use for the applicator 10. The distal end may be flared, as shown, and its largest cross-section can also come in a wide range of sizes, preferably from about 0.5 to about 5 cm, generally less than about 2 cm, but it is most preferably chosen to be larger than the width of the wound to be closed. The configuration of the opening 13 can be a narrow elliptical or rectangular slot or other configuration suited for the end use. The reservoir 11 is preferably compressible or collapsible to allow for greater control in the rate at which the adhesive is expressed from the opening 13. The reservoir may comprise any suitable material, including metal (such as metal foil) and plastic and combinations thereof.

In one embodiment, the distal opening 13 of the applicator tip 14 has a shape like that shown in FIG. 4. During application, the flattened side is placed towards the skin to maximize the area of applicator-skin contact. This semi-elliptical or other concave shape of the opposing side of opening 13 results in application of a rounded strip of adhesive as the tip 14 is drawn across the surface of the skin. Center thickness on the order of at least about 2 times and often as much as about 3 or about 5 times or greater the edge thickness are preferably achieved. This shape optimizes adhesive tensile strength across the top of the wound. In general the combination of thickness and inherent adhesive characteristics for a particular adhesive should be optimized to provide both a sufficient bond to the skin as well as sufficient tensile strength to resist tearing under reasonably anticipated forces encountered by normal activities of the patient. Although this tip shape has advantages in accordance with some embodiments, use of tips having other shapes and configurations are contemplated, including a dropper tip, tube, opening between two sheets of foil, and the like.

The applicator tip of the container-applicator can further comprise a removable or breakable sealing tip. One embodiment of a breakable sealing tip, which can be used for a single-use device, is shown in FIG. 5. The applicator cap 15 forms a solid covering for the opening of the applicator tip (14 in FIG. 5). The applicator cap 15 is preferably of the same material as the applicator tip 14, the two parts meeting at a breakline 16. In one embodiment in which the tip 14 and cap 15 are integrally formed, the breakline 16 is characterized by scoring or other means which weakens the junction and allows the two parts to be easily separated by grasping the two parts (12 and 15) in either hand and bending or tearing the pieces apart to expose the opening 13 in the applicator tip 12 through which the adhesive is expressed. The cap 15 can alternatively be press fit or threadably engaged within or over the distal tip 14 and retained by friction as will be apparent to those of skill in the art.

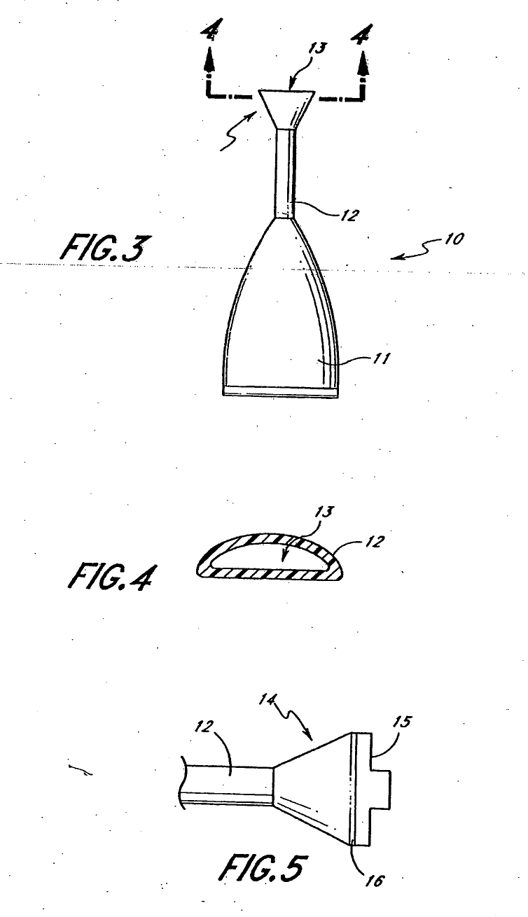

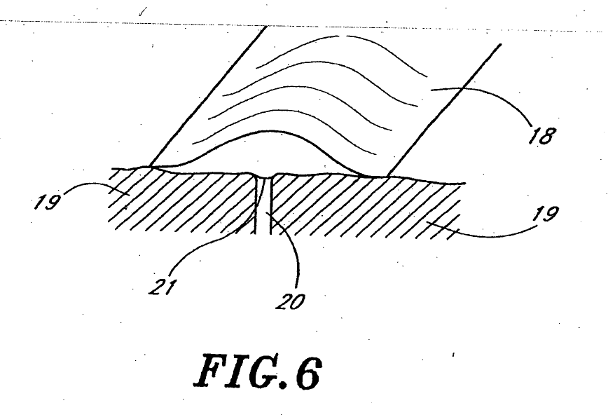

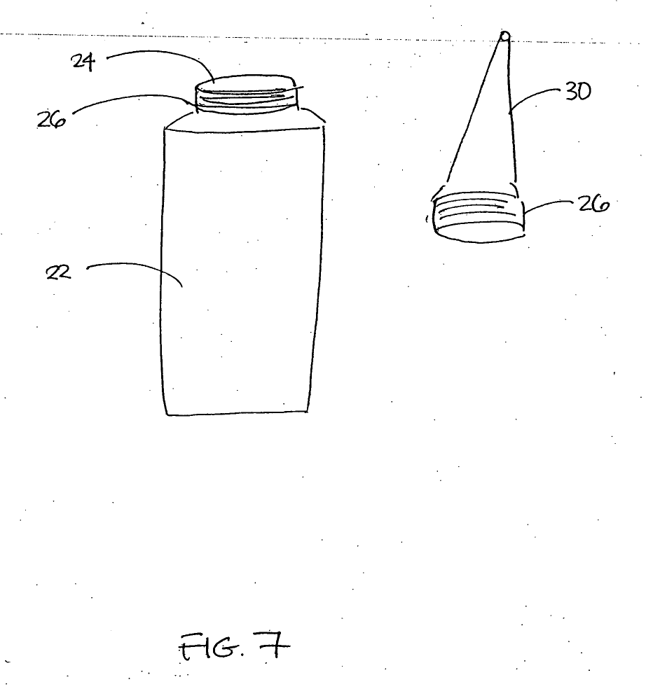

Alternatively, a single-use device can comprise a reservoir wherein the opening through which the adhesive flows is covered with a peelable or puncturable plastic film or metal foil. In one such embodiment, the foil or film is peeled back or pierced prior to positioning the applicator and sealing the wound. In another embodiment, the applicator portion has a proximally extending point or projection which pierces the foil or film as it is threaded onto or otherwise secured to the reservoir. As shown in FIG. 7, a certain embodiment comprises inert collapsible or compressible metal foil tubes with long-necked applicators. Adhesive material is easily delivered from the tube 22 by puncturing a seal 24 of a screw 26 on top of the container-applicator. A long-necked screw 28 with a cannula 30 is positioned tightly with the screw 26 of the container-applicator. The adhesive material can be expressed through an orifice of the cannula and applied evenly with a beveled length of the cannula to a desired thickness of adhesive material. The long neck applicator can be catheter like device with orifices of 20-25 gauge and 1 in in length.

One embodiment of container-applicator is a single-use, sterile wound closure device. Preferably such a device has a pierceable or removable tip seal. The container portion of the preferred single-use wound closure device is sized to hold preferably from about 0.2 to about 1.0 grams, more preferably about 2 g of tissue adhesive, depending upon the intended use. The container can be of any of a variety of standard container shapes, and is preferably compressible or collapsible so that the user may control the rate at which the adhesive contained therein is expressed by varying the pressure exerted on the walls of the container.

The single-use sterile wound closure device is prepared by first taking a clean container that will serve as the reservoir and filling it with adhesive. The reservoir is then sealed. Sealing the reservoir is preferably done by affixing an applicator tip with a removable seal to the reservoir, or by securing a pierceable septum to the container. The container-applicator, with the adhesive sealed inside, is then sterilized by methods known to those skilled in the art which can be used on the materials from which the container-applicator is made and which will also not react with the adhesive.

In the alternative, the pieces which comprise the container-applicator can be pre-sterilized, and the device filled and sealed in a sterile or ultra clean environment. This is potentially a viable method, as a preferred adhesives comprising cyanoacrylates are generally not supportive of the growth of microorganisms.

The use of reusable coverings for applicators or applicator openings, such as caps, plugs, valves, or the like are also contemplated. Use of this type of covering would allow a container or container-applicator to be used several times before it is discarded.

The containers, applicators, and container-applicators disclosed above can be used alone, in combination with a support structure, such as a piece of cloth, gauze or mesh, or in addition to some other securing means such as sutures or staples. Support structures can provide an extra measure of strength and protection for the wound, while use of a adhesive with sutures or staples can reinforce and thoroughly seal the joint to help prevent rupture, protect the joint from abrasion, or keep it free of debris. Similarly, for a deep or penetrating wound or surgical incision, the innermost tissues can be joined by dissolvable sutures while the exterior surface is joined using adhesives according to the preferred embodiments.

Closure of a wound can also be effected by the use of a device comprising a support structure impregnated with adhesive. In such a device, the support structure, comprising cloth or gauze, has a sufficient quantity of adhesive imbedded therein to allow for closure and sealing of a wound. Preferably, each device is individually sealed within air-and-water-tight packaging such as a plastic or foil pouch until use. Although the application and use of such a device would be very similar to a conventional adhesive bandage, it has several advantages. The adhesive impregnated support structure will adhere to the wound for a much longer time than a conventional adhesive bandage and provide a better barrier to water, dirt, and abrasion. The adhesive impregnated support structure would be especially suitable for use on children, as it would keep the wound cleaner and prevent the child from disturbing the wound and hampering the healing process.

Alternatively, an adhesive material according to preferred embodiments may be applied over an adhesive tape. The adhesive material is preferably applied over the adhesive tape in a quantity sufficient to penetrate the tape and provide additional securement force to maintain closure of the wound. The adhesive material covering can also aid in reducing lifting or separating of the tape from the skin and serve as a barrier to moisture or contamination.

Generally the methods of the preferred embodiments proceed by delivering the appropriate adhesive to the percutaneous opening. Following application, the adhesive is allowed to set up. Methods of the preferred embodiments can optionally include steps of bringing the sides of the wound into opposition, applying another closure modality to be used in conjunction with the adhesive, and/or holding the surfaces together until the adhesive has adequate strength to hold the opening closed.

In a preferred embodiment, the adhesive takes on a bell-curve type shape as shown in FIG. 6 following application. This shape is advantageous in that it places the thickest part of the layer, and thus the strongest part of the layer, over the opening in the skin to provide enhanced resistance to tearing, rupture, or other stress or damage to the opening following closure. The added protection provided by a thicker layer can aid in speeding the healing process and allowing for a minimum of stretching of the wound as it heals, which may help minimize scarring. The adhesive can be applied such that the thickness over the percutaneous opening is at least about 0.1 mm, often from about 0.5 mm to about 4 mm thick, or from about 1.0 to about 2.5 mm thick.

In one embodiment, an adhesive is used for which the polymerization process of the adhesive is enhanced by the presence of water or a basic substance. Prior to application of the adhesive, water is optionally placed on the skin in the general area of the wound or opening, including a spraying or misting water or saline on the area, or wiping the area with an alcohol/water prep pad. The water which remains on the surface may increase the rate at which the adhesive sets up on the wound. However, it should be noted that in some cases the presence of additional water may impair crosslinking and the polymerization and eventual tensile strength of the adhesive.

Initiators, cross-linkers, catalysts, and other compounds which aid an adhesive in setting up can be applied in a similar manner, provided that they would not irritate the open wound, or cause other undesirable side effects.

When applying adhesive of the preferred embodiments, the surfaces of the wound, laceration, percutaneous incision, or the like intended for closure are brought in contact with each other by use of the fingers, forceps, or a similar device. A sufficient amount of adhesive is delivered to the surface so that proper sealing and closure retention will occur. When sealing the joint formed by the sides of the wound, laceration, or percutaneous incision, the adhesive is applied to the exterior surface of the wound and allowed to polymerize so that it forms a film over the entire wound. Preferably, adhesives are applied in a manner to minimize the amount of adhesive which seeps between the edges of a wound. The amount of adhesive to apply in any given case, and thus the area and thickness of the resulting film, can depend on several factors including placement of the wound on the body, depth of the wound, tissue sensitivity to the adhesive, and the like. Adhesive can be applied alone or in combination with a support structure or other securing means such as sutures. Through routine experimentation, however, one of skill in the art will be able to exercise clinical judgment to determine an appropriate quantity of adhesive to provide effective closure for a particular procedure.

Methods of the preferred embodiments are preferably directed toward closing and sealing a wound by sealing and securing together adjacent tissues, such as opposing pieces of skin, in a patient. The need for closure of such a wound can arise during surgical procedures, as a result of percutaneous incision. The need can also arise as a result of traumatic injury resulting in a laceration or other wound which breaks the skin.

Generally, a method of closing a wound, laceration, percutaneous incision, or the like proceeds by first assessing what type of closure or combination of closures is proper for a wound given factors such as the size, depth, and location of the wound as well as an assessment of the overall needs and requirements of the patient. Such assessments are routinely done by those skilled in the medical arts. In a non-clinical setting, the assessment step will likely be much more cursory.

Next, a suitable formulation of adhesive, an applicator, and a method of application are chosen. These three choices are somewhat interconnected, as the choice of a particular applicator constrains the method of application, and a particular formulation of adhesive can constrain the type of applicator or method of application which can be used, and vice-versa.

The choice of a suitable formulation of wound closure adhesive, as disclosed herein, can depend upon characteristics of a adhesive such as its viscosity, biodegradability and rate thereof, resulting tensile strength upon polymerization, flexibility when polymerized, histotoxicity, and polymerization rate. Specific characteristics can be desired to fit clinical needs as dictated by factors such as the size of the wound, the amount and rate of bleeding from the wound, the location of the wound on the body, and potential stress on the sealed wound.

The choice of applicator and method of application can, in part, be determined by factors such as the composition, viscosity, and polymerization time of the adhesive, and the geometry, size and placement of the application site. Such a choice can also be constrained by the tools and devices available to the user. Examples of preferred applicators are disclosed above and examples of preferred methods of application are described below.

Next, the wound may need to be prepared before closure. Activities involved in wound preparation are highly situational, but are routinely done by those skilled in medicine, nursing, and related arts. Wound preparation can involve tasks, such as removal of debris, dirt, oil, or excess tissue from the wound, application of pressure or similar measures to bring about the cessation of bleeding, cleansing the wound, application of an antimicrobial preparation, use of additional closure means such as sutures, and other such tasks. In a non-clinical setting, the patient or use can also perform some of these same tasks.

If the surfaces of the wound naturally pull apart, it can be advantageous to bring the two surfaces into contact with each other and align them by use of the fingers, support structure, forceps or other suitable medical instrument. In such a case, the two surfaces are preferably held together as the adhesive is applied and afterwards until sufficient polymerization has taken place to allow the closure to be self-supporting. Alternatively, the two surfaces can be brought together by sutures, staples, tape or other securing means and then further sealed by application of a chosen adhesive. Such methods can allow for eventual scarring of the opening to be minimized. In wounds for which the skin is not separated, this step can be skipped.

The chosen adhesive is then applied using the applicator and method chosen in an earlier step. The entirety of adhesive application is preferably done within a limited period of time, as the strength of the closure formed by two or more successive applications of adhesive (wherein one application has been allowed to polymerize before the next application) may not be as strong as the closure formed by one application allowed to polymerize to form a single layer on the skin surface. Adhesive is applied in a quantity sufficient to effect wound closure and sealing. More can be applied, if desired, to increase the strength of the closure as discussed above, or likewise a support structure may be applied. Determination of quantity of adhesive applied can be determined by routine experimentation and exercise of clinical judgment. Specific methods of application involving the use of container-applicators are discussed in the paragraphs which follow.

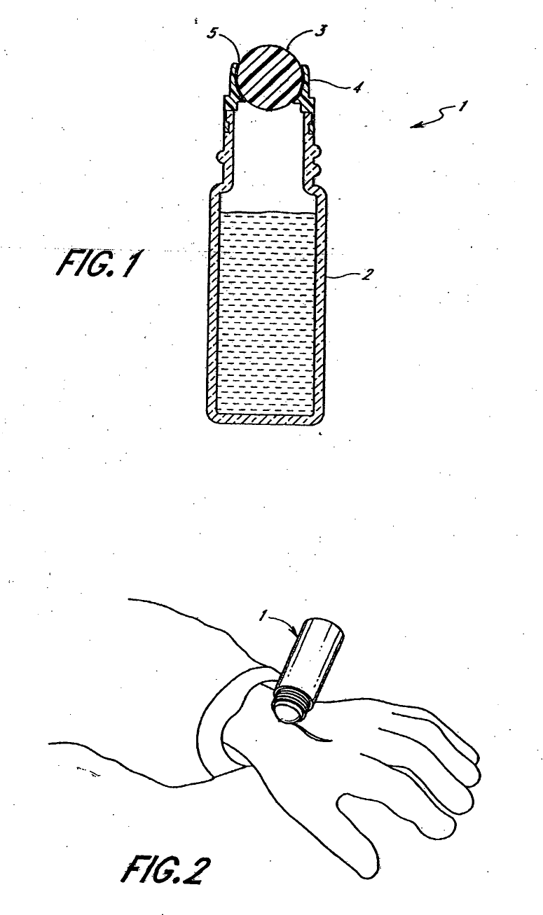

One specific method of application is that involving the use of the rollerball container-applicator pictured in FIG. 1. To use this container-applicator, first any sealing means is removed or broken. Then, as depicted in FIG. 2, the container is tipped so that the rollerball is pointing in a generally downward direction and the bottom of the reservoir portion is pointing in a generally upward direction. Such orientation of the container-applicator facilitates the flow of adhesive towards the rollerball applicator portion through which it can then be applied to the wound. Preferably the adhesive is applied by moving the container-applicator back and forth over the surface of the wound and surrounding skin areas while keeping the rollerball in contact with the wound at all times. Although a back and forth movement is preferred, any movement of the applicator which serves to deliver the adhesive to the intended site without disturbing the wound itself is contemplated.

If the reservoir portion of the container-applicator is compressible or collapsible, the rolling of the applicator over the surface of the skin can be accompanied by squeezing or otherwise compressing the walls of the reservoir. With such a collapsible or compressible reservoir, the rate of flow of adhesive and therefore the amount of adhesive delivered, is proportional to the amount of pressure applied to the walls of the reservoir. The quantity and rate of adhesive delivery can thus be controlled by the user.

Another specific method of application is that using a container-applicator of the type depicted in FIG. 3. To apply adhesive, first any sealing means such as a foil seal, peel-away thin film or breakable tip is punctured or removed to allow for flow of adhesive. The applicator tip is preferably placed on or slightly above the surface of the wound to be sealed. If a semi-elliptical tip is used, such as that pictured in FIG. 4, the flattened side is preferably placed closest to the skin. The adhesive is then allowed to flow through the applicator and onto the surface of the skin forming a profile such as shown in FIG. 6. Preferably, the reservoir portion comprises collapsible or compressible walls such that the user may exert pressure on the walls to facilitate the delivery of adhesive to the skin, and thus control the rate at which the adhesive is expressed from the applicator tip. The tip is moved over the surface of the skin, following the contours of the wound, resulting in the deposition of a strip of adhesive on the skin covering the wound. Additional strips may be laid down in a similar manner to thicken or expand the area of adhesive coverage.

In accordance with another embodiment, the reservoir is provided as a separate component from the applicator tip. In this embodiment, the reservoir is provided with a pierceable seal or septum, such that a unit volume of adhesive can be sealed within the reservoir. Pierceable septums or seals comprising silicone, other polymeric materials known in the medical industry, as well as metal foils or thin polymeric films may be utilized, as will be apparent to those of skill in the art in view of the nature of the complimentary piercing structure on the applicator tip.

The detachable applicator tip comprises an applicator surface on a distal side thereof, and a cannula, needle or other piercing structure projecting proximally from a proximal side thereof. A retention structure is preferably also provided, for securing the applicator tip to the reservoir. In one embodiment, the retention structure is an axially extending annular flange having a thread on the radially inwardly or outwardly facing surface thereof, for threadably engaging the top of the reservoir. Any of a variety of other retention structures can be utilized, as will be apparent in view of the disclosure herein.

Prior to use at the clinical site, the applicator tip is secured to the reservoir such that the proximally extending piercing member on the proximal side of the applicator tip pierces the septum or other seal on the reservoir, thereby placing the contents of the reservoir in fluid communication with the distal applicator surface. This embodiment is particularly suited for a one-time use disposable device. The applicator surface can be of any of a variety of structures disclosed elsewhere herein, such as a rollerball, or a specially configured opening such as a slot, for expressing a thin layer of sealing adhesive over the surface of the tissue on either side of a wound.

Any of the foregoing methods can be combined with the application of a support structure, such as gauze. A layer of adhesive is first applied to the wound, onto which gauze or other support structure is affixed, the adhesive acting to secure the gauze in place. More adhesive may then be applied over the gauze to further secure it and strengthen the closure. In the alternative, gauze can be first placed over the wound and then covered and secured to the wound by subsequent application(s) of adhesive as described above. In either case, alternate layers of adhesive and gauze may be applied to form a flexible, reinforced structure which effects closure of the wound and sealing.

As an alternative to the method discussed above, a prepackaged adhesive-impregnated support structure can be applied to the wound to achieve closure. Such a device, as described above, is preferably packaged in a sealed pouch and comprises a support structure, such as a section of cloth, that is saturated with a quantity of adhesive sufficient to allow for attachment of the support structure and effect closure of a wound or section of a wound of a size corresponding to the size of the support structure. Closure of a wound using such a device is somewhat comparable to using a common adhesive bandage and is particularly well-suited for non-clinical use. First, the pouch containing the device is opened and the device removed therefrom. The device is then placed over the surface of the wound and then pressed into place to ensure good contact between the device and the skin. If additional coverage is required or desired, additional devices may be applied. When more than one device is used, they are preferably applied within a short time of each other so that they polymerize at nearly the same time.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods may be performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Similarly, the various features and steps discussed above, as well as other known equivalents for each such feature or step, can be mixed and matched by one of ordinary skill in this art to perform methods in accordance with principles described herein.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein, but instead by reference to claims attached hereto.

1. A tissue adhesive, comprising:
about 70-95% by volume of a mixture of first and second cyanoacrylate monomers, each having the general formula (I): 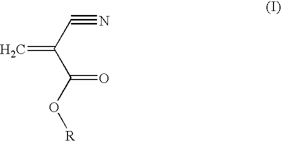
wherein R is alkyl; and
the first cyanoacrylate monomer has at least 3 carbon atoms and the second cyanoacrylate monomer has no more than 10 carbon atoms;
about 1-15% by volume cyanoacrylate polymer; and
about 2-15% by volume plasticizer.

2. A tissue adhesive according to claim 1, wherein the viscosity is within the range of from about 100 to about 50,000 centipoise.

3. A tissue adhesive according to claim 1, wherein the viscosity is within the range of from about 100 to about 5,000 centipoise.

4. A tissue adhesive according to claim 1, wherein the adhesive comprises about 80-90% cyanoacrylate monomer, about 5-10% cyanoacrylate polymer, and about 5-10% plasticizer.

5. A tissue adhesive according to claim 1, wherein the first cyanoacrylate monomer is octylcyanoacrylate and the second cyanoacrylate monomer is butylcyanoacrylate.

6. A tissue adhesive according to claim 1, wherein the monomer mixture comprises about 50% to about 95% of the first cyanoacrylate monomer and about 5% to about 50% of the second cyanoacrylate monomer.

7. A tissue adhesive according to claim 7, wherein the monomer mixture comprises about 65% to about 75% of the first cyanoacrylate monomer and about 25% to about 35% of the second cyanoacrylate monomer.

8. A tissue adhesive according to claim 7, wherein the first cyanoacrylate monomer is octylcyanoacrylate and the second cyanoacrylate monomer is butylcyanoacrylate.

9. A tissue adhesive according to claim 1, wherein the cyanoacrylate polymer comprises poly(octylcyanoacrylate).

10. A tissue adhesive according to claim 1, wherein the plasticizer is tributyl citrate.

11. A method of closing a percutaneous opening, having a first dermal surface on a first side of the opening and a second dermal surface on a second side of the opening and generally coplanar with the first dermal surface, the method comprising the step of:
applying an adhesive layer across at least a portion of the first and second dermal surfaces and spanning the opening,
wherein the adhesive comprises one or more cyanoacrylate monomers, cyanoacrylate polymer, and plasticizer; and
wherein the adhesive exhibits a sufficient viscosity to substantially prevent flow of the adhesive into the opening.

12. A method as in claim 11, wherein the viscosity is within the range of from about 100 to about 50,000 centipoise.

13. A method as in claim 11, wherein the viscosity is within the range of from about 100 to about 5,000 centipoise.

14. A method as in claim 11, wherein the viscosity is at least about 100 centipoise.

15. A method as in claim 11, wherein the adhesive comprises about 70-95% cyanoacrylate monomer, about 1-15% cyanoacrylate polymer, and about 2-15% plasticizer.

16. A method as in claim 15, wherein the adhesive comprises about 80-90% cyanoacrylate monomer, about 5-10% cyanoacrylate polymer, and about 5-10% plasticizer.

17. A method as in claim 11, wherein the cyanoacrylate monomer comprises a mixture of octylcyanoacrylate and butylcyanoacrylate.

18. A method as in claim 17, wherein the cyanoacrylate monomer comprises about 60% to about 80% octylcyanoacrylate and about 20% to about 40% butylcyanoacrylate.

19. A method as in claim 17, wherein the cyanoacrylate monomer comprises about 65% to about 75% octylcyanoacrylate and about 25% to about 35% butylcyanoacrylate.

20. A method as in claim 11, wherein the cyanoacrylate polymer comprises poly(octylcyanoacrylate).

21. A method as in claim 11, wherein the plasticizer is tributyl citrate.

22. A method as in claim 11, wherein the adhesive layer has a thickness over the opening of at least 1 millimeter.

23. A method of closing and sealing a joint formed between a tissue of a patient and a second surface, comprising:
positioning a tissue of a patient adjacent to a second surface;
securing the tissue to the second surface using a primary closure modality to form a joint;
delivering an adhesive comprising cyanoacrylate monomer, cyanoacrylate polymer, and a plasticizer to the joint; and
permitting the adhesive to polymerize.

24. A method as in claim 23, wherein the second surface is an autologous tissue, allograph tissue, animal tissue, biologically compatible metal, polymeric material, or prosthetic device.

25. A method as in claim 23, wherein the primary closure modality is sutures or staples.

26. A method as in claim 23, wherein the tissue and the second surface are adjacent surfaces of the skin of a patient.

27. A method of closing and sealing a wound in a patient, comprising the steps of:
identifying a percutaneous wound having first and second sides;
delivering a layer of wound closure adhesive comprising one or more cyanoacrylate monomers, cyanoacrylate polymer, and plasticizer to the surface of skin on each of the first and second sides and across the wound in a quantity sufficient to retain closure and sealing of said wound; and
restraining adhesive from entering the wound.

28. A method as in claim 27, wherein the restraining step comprises providing the wound closure adhesive with a sufficient viscosity to extend in a layer across the wound, while substantially preventing the adhesive from entering the wound.

29. A method as in claim 28, wherein the viscosity is at least 100 centipoise.

30. A method of covering a trauma to an outer layer of a skin in a patient comprising:
delivering an adhesive comprising cyanoacrylate monomer, cyanoacrylate polymer, and a plasticizer to a surface of the skin covering an area of the trauma to the outer layer of the skin in a quantity sufficient to cover said trauma; and
permitting the adhesive to polymerize.

31. A method as in claim 30, further comprising applying an aqueous solution to the trauma prior to delivering the adhesive.

32. A method as in claim 30, wherein the adhesive has a prepolymerization viscosity of at least about 100 centipoise.

33. A method as in claim 30, wherein the trauma is selected from the group consisting of abrasions, scrapes, burns, blisters, bedsores, ulcers, chapping, and chafing.
